(19) United States
(12) Patent Application Publication
(10) Pub. No.: US 2025/0018209 A1
(43) Pub. Date: Jan. 16, 2025

(54) APPARATUSES, METHODS, AND SYSTEMS FOR TREATING SPINAL TUMORS WITH TUMOR TREATING FIELDS

(71) Applicant: Novocure GmbH, Root D4 (CH)

(72) Inventor: Ariel NAVEH, Haifa (IL)

(73) Assignee: Novocure GmbH, Root D4 (CH)

(21) Appl. No.: 18/770,132

(22) Filed: Jul. 11, 2024

Related U.S. Application Data
(60) Provisional application No. 63/526,332, filed on Jul. 12, 2023.

(51) Int. Cl. A61N 1/40

(52) U.S. Cl. CPC A61N 1/40

(57) ABSTRACT

A method of applying tumor treating fields to a subject includes: locating a plurality of transducers on the subject, the plurality of transducers being located to focus electric fields around a spinal area of the subject; and alternately inducing a first electric field for a first set of the transducers and a second electric field for a second set of the transducers.

### CROSS-REFERENCE TO RELATED APPLICATIONS

This Application claims priority to U.S. Provisional Application No. 63/526,332 filed Jul. 12, 2023, the contents of which is incorporated by reference herein in its entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. In current commercial systems, TTFields are induced non-invasively into a region of interest by electrode assemblies (e.g., arrays of capacitively coupled electrodes, also called electrode arrays, transducer arrays or simply “transducers”) placed on a subject's body and applying AC voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIG. 1 (which includes FIGS. 1A and 1B) illustrates an exemplary layout of transducers to deliver TTFields.

FIG. 2 illustrates a laminectomy model for use in a computer simulation using the exemplary layout of transducers of FIG. 1.

FIG. 3 illustrates simulation results using the exemplary layout of transducers of FIG. 1.

FIG. 4 (which includes FIGS. 4A and 4B) illustrates an exemplary layout of transducers to deliver TTFields.

FIG. 5 illustrates simulation results using the exemplary layout of transducers of FIG. 4.

FIG. 6 illustrates an exemplary layout of transducers to deliver TTFields.

FIG. 7 illustrates a laminectomy model for use in a computer simulation using the exemplary layout of transducers of FIG. 6.

FIG. 8 illustrates simulation results using the exemplary layout of transducers of FIG. 6.

FIG. 9 (which includes FIGS. 9A, 9B, and 9C) illustrates an exemplary layout of transducers to deliver TTFields.

FIG. 10 illustrates a laminectomy model for use in a computer simulation using the exemplary layout of transducers of FIG. 9.

FIG. 11 illustrates simulation results using the exemplary layout of transducers of FIG. 9.

FIG. 12 depicts an example apparatus to apply alternating electric fields (e.g., TTFields) to a subject.

FIGS. 13A and 13B depict schematic views of exemplary designs of a transducer for applying alternating electric fields.

FIG. 14 depicts an example placement of transducers on a subject's head.

FIG. 15 depicts an example computer apparatus according to one or more embodiments described herein.

FIG. 16 depicts an example method for determining transducer locations for delivering TTFields to a subject according to one or more embodiments described herein.

### DESCRIPTION OF EMBODIMENTS

This application describes exemplary apparatuses, methods, and systems for treating spinal tumors with alternating electric fields (e.g., TTFields).

Certain types of cancers tend to develop spinal metastases. These metastases apply stress on the spinal cord, leading to neurologic dysfunctionalities with different levels of severity. A laminectomy is usually performed on these patients, albeit with a high rate of recurrent tumors in the space left behind the lamina.

The inventor has discovered layouts for transducers to deliver sufficiently high levels of alternating electric fields (e.g., TTFields) to tumors growing into the resected laminae. The discovered layouts aid to deliver sufficiently high levels of TTFields outside the spinal cord itself and not solely treat astrocytoma tumors within the spinal cord using TTFields.

The inventor has discovered layouts that are able to treat spinal tumors within three different regions of the spinal cord: 1) vertebrae T2-L4; 2) vertebrae C1-T2; and 3) vertebrae L4-L5.

The inventor has further discovered the impact of different materials used as implants on the field distribution of the TTFields. Titanium is widely used as a material for spinal implants. The inventor has discovered that titanium can influence the current flow and hence the field distribution of TTFields. This impact on the current flow may likely be due to the high conductivity of titanium. With a titanium implant located in the region of interest for delivery of TTFields, the TTFields delivered to the region of interest tend to move to other areas of the subject via the titanium implant and away from the region of interest, which results in the dosage delivered to the region of interest being less than a desired dosage.

To counter the highly conductive nature of titanium, the inventor has discovered that using polyether ether ketone (PEEK) as the material for the medical implant may increase the field distribution of the TTFields compared to using titanium as the material for the medical implant. Further, using a non-conductive material or a substantially non-conductive material for the medical implant may increase the field distribution of the TTFields compared to using titanium as the material for the medical implant. For example, an implant having a substantially non-conductive material may include: a non-conductive material “core” coated with a thin layer of conductive material; a small amount of conductive material (e.g., a rod or other support structure) encased in a non-conductive material; or a non-conductive material having less than 50% (or less than 40%, 30%, 20%, 10%, 5%, 2%, or 1%, or values therebetween) of its surface area being a conductive material. With an implant at least partially including a non-conductive material (e.g., PEEK) located in the region of interest for delivery of TTFields, the TTFields delivered to the region of interest may tend to stay in the region of interest and not move away from the region of interest, which may result in the dosage delivered to the region of interest being the same as or substantially the same as a desired dosage. With an implant consisting entirely of a non-conductive material (e.g., PEEK), the results may be improved.

As such, increased field intensity for delivered TTFields may be obtained when using PEEK as the material of the implant compared to widely used titanium as the material of the implant. Moreover, increased field intensity for delivering TTFields may be obtained when using a non-conductive material or a substantially non-conductive material for the implant compared to a conductive-material for the implant.

### Thoracic-High Lumbar Region—Vertebrae T2-L4

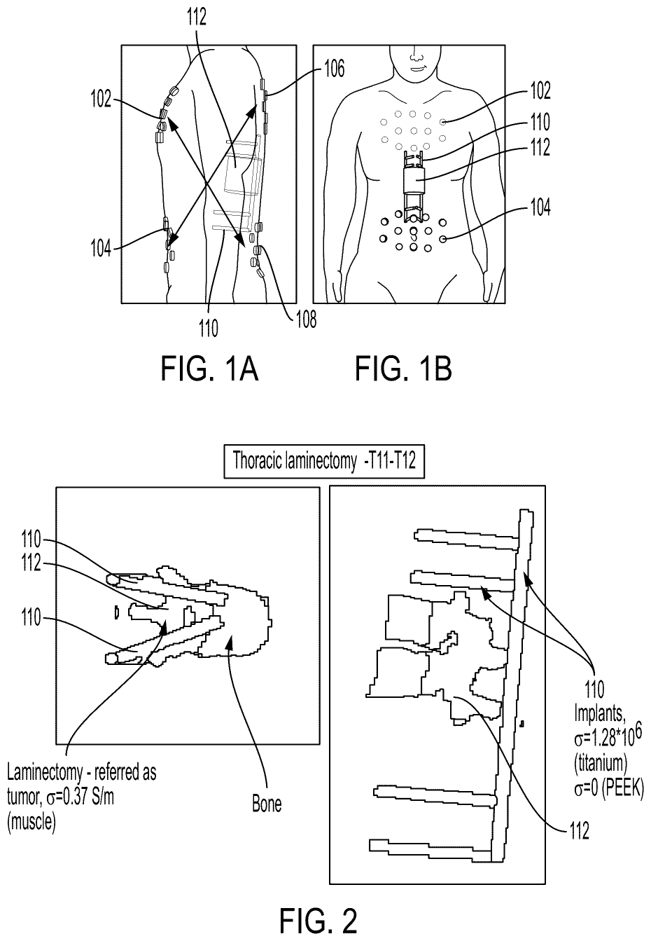

FIG. 1 (which includes FIGS. 1A and 1B) illustrates an exemplary layout of transducers to deliver TTFields to a tumor in a thoracic-high lumbar region of a subject and/or in a region around vertebrae T2-L4 of a subject.

FIG. 1A illustrates a side view of the subject, and FIG. 1B illustrates a front view of the subject. The exemplary layout includes four transducers 102, 104, 106, and 108: a first transducer 102 located on the upper chest on the front of the subject; a second transducer 104 located on the abdomen on the front of the subject; a third transducer 106 located on the upper back of the subject; and a fourth transducer 108 located on the lower back of the subject. When applying TTFields to the subject, AC voltage may be alternately applied to a first pair of transducers 102 and 108 and to a second pair of transducers 104 and 106. In the example of FIG. 1, each of the first, second, third, and fourth transducers has 13 electrode elements. In some embodiments, each of the first, second, third, and fourth transducers may have less than or more than 13 electrode elements. In some embodiments, the first, second, third, and fourth transducers may have a different number of electrode elements.

Each of the first, second, third, and fourth transducers may be configured in a variety of shapes and positions to generate an electric field of the desired configuration, direction, and intensity at a target volume to focus treatment. Each of the first, second, third, and fourth transducers may be configured to deliver two perpendicular field directions through a volume of interest. The arrows in FIG. 1A indicate the TTfields generated within the treated tumor for each of the two pairs of transducers.

FIG. 2 illustrates a laminectomy model for use in a computer simulation using the exemplary layout of transducers of FIG. 1. The model appears pixelated due to the discrete computation model used. The electric distribution generated between the transducers may be simulated to determine a desired treatment therapy. For the simulation, the model included a “virtual laminectomy” on the subject's spine. In the resected region, a mock tumor 112 was inserted in the model to mimic typical metastatic growth in the region. The mock tumor 112 was referred to as muscular tissue in the software simulation, as it presents relatively high conductivity, typical to tumors. In other words, the mock tumor 112 may be assigned the dielectric properties typical to tumors. For the simulation, the resected vertebrae included thoracic vertebrae T11 to T12, the mock tumor 112, and an implant 110. For the simulation, two materials were tested, namely titanium and PEEK. In some embodiments, a “mix material” implant 110 may be simulated. For example, an implant 110 with a non-conductive material “core” coated with a thin layer of conductive material, or the inverse, may be tested in the simulation.

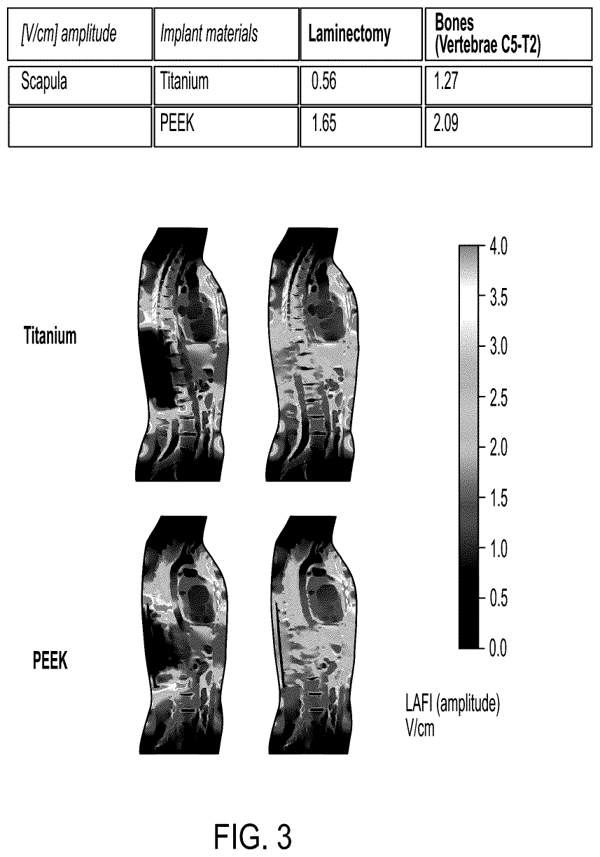

FIG. 3 illustrates the simulation results using the exemplary layout of transducers of FIG. 1. As can be seen, the exemplary layout of transducers provides high dosages of TTFields for both the laminectomy region and a region around vertebrae C5-T2. Further, using PEEK as the material for the implant 110 provides higher dosages of TTFields than using titanium as the material for the implant 110.

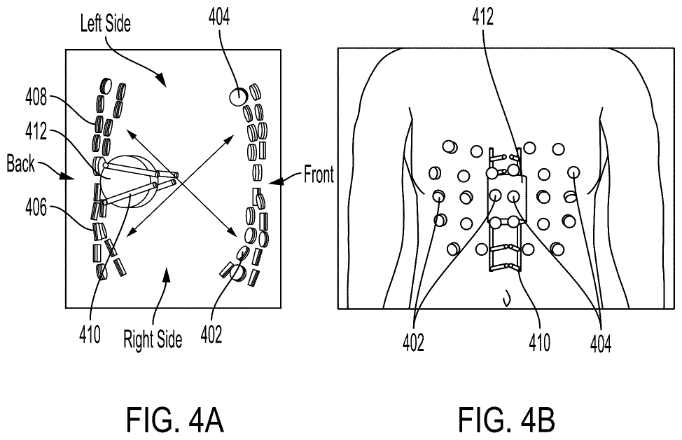

FIG. 4 (which includes FIGS. 4A and 4B) illustrates an exemplary layout of transducers to deliver TTFields to a tumor in the thoracic-high lumbar region of a subject and/or in a region around vertebrae T2-L4 of a subject. FIG. 4A illustrates a top view of the four transducers with the subject redacted, and FIG. 4B illustrates a front view of the subject, each including an implant 410 and mock tumor 412. The exemplary layout includes four transducers, including: a first transducer 402 located on the right side of the mid-torso on the front of the subject; a second transducer 404 located on left side of the mid-torso on the front of the subject; a third transducer 406 located on the right side of the mid-torso on the back of the subject; and a fourth transducer 408 located on left side of the mid-torso on the back of the subject. When applying TTFields to the subject, AC voltage is alternately applied to a first pair of transducers 402 and 408 and to a second pair of transducers 404 and 406. In the example of FIG. 1, each of the first, second, third, and fourth transducers have 13 electrode elements. In some embodiments, each of the first, second, third, and fourth transducers may have less than or more than 13 electrode elements. In some embodiments, the first, second, third, and fourth transducers may have a different number of electrode elements.

Each of the first, second, third, and fourth transducers may be configured in a variety of shapes and positions to generate an electric field of the desired configuration, direction, and intensity at a target volume to focus treatment. Each of the first, second, third, and fourth transducers may be configured to deliver two perpendicular field directions through a volume of interest. The arrows in FIG. 4A indicate the TTfields generated within the treated tumor for each of the two pairs of transducers.

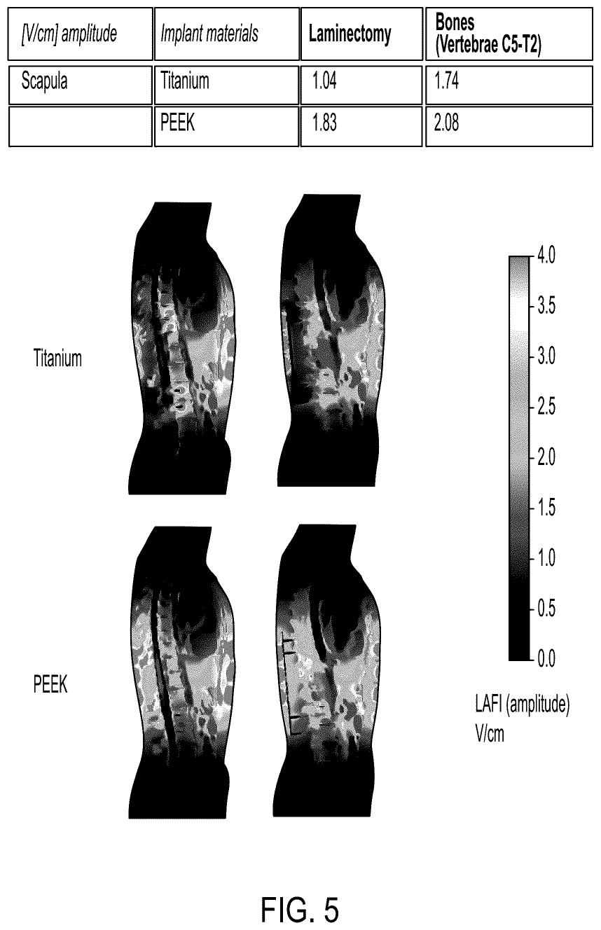

Using the laminectomy model of FIG. 2, a computer simulation using the exemplary layout of transducers of FIG. 4 was performed. The electric distribution generated between the transducers may be simulated to determine a desired treatment. FIG. 5 illustrates the simulation results. As can be seen, the exemplary layout of transducers provides high dosages of TTFields for both the laminectomy region and a region around vertebrae C5-T2.

FIG. 5 illustrates the simulation results using the exemplary layout of transducers of FIG. 4. As can be seen, the exemplary layout of transducers provides high dosages of TTFields for both the laminectomy region and a region around vertebrae C5-T2. Further, using PEEK as the material for the implant 410 provides higher dosages of TTFields than using titanium as the material for the implant 410.

### Cervical Region-Cervical-High Thoracic Region-Vertebrae C1-T2

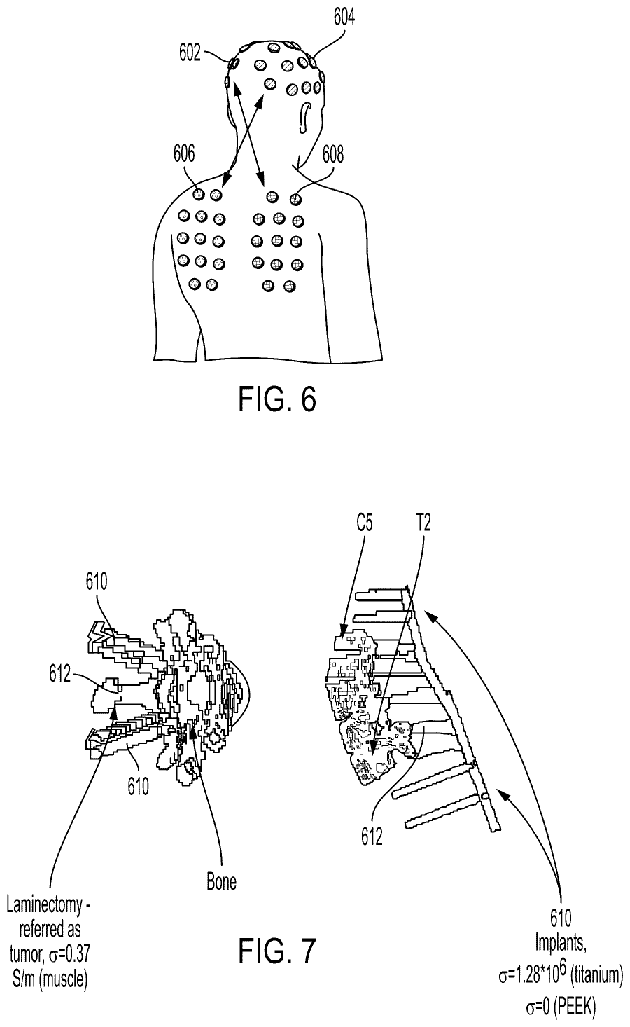

FIG. 6 illustrates an exemplary layout of transducers to deliver TTFields to a tumor in a cervical region of a subject, in a cervical-high thoracic region of a subject, and/or in a region around vertebrae C5-T2 of a subject.

FIG. 6 illustrates a perspective view of the back right side of the subject. The exemplary layout includes four transducers, including: a first transducer 602 located on the left side of the head of the subject; a second transducer 604 located on the right side of the head of the subject; a third transducer 606 located on the upper left back of the subject; and a fourth transducer 608 located on the upper right back of the subject. When applying TTFields to the subject, AC voltage is alternately applied to a first pair of transducers 602 and 608 and to a second pair of transducers 604 and 606. In the example of FIG. 1, each of the first, second, third, and fourth transducers have 13 electrode elements. In some embodiments, each of the first, second, third, and fourth transducers may have less than or more than 13 electrode elements. In some embodiments, the first, second, third, and fourth transducers may have a different number of electrode elements.

Each of the first, second, third, and fourth transducers may be configured in a variety of shapes and positions to generate an electric field of the desired configuration, direction, and intensity at a target volume to focus treatment. Each of the first, second, third, and fourth transducers may be configured to deliver two perpendicular field directions through a volume of interest. The arrows in FIG. 6 indicate the TTfields generated within the treated tumor for each of the two pairs of transducers.

FIG. 7 illustrates a laminectomy model for use in a computer simulation using the exemplary layout of transducers of FIG. 6. The model appears pixelated due to the discrete computation model used. The electric distribution generated between the transducers may be simulated to determine a desired treatment. For the simulation, the model included a “virtual laminectomy” on the subject's spine. In the resected region, a mock tumor 612 was inserted in the model to mimic typical metastatic growth in the region. The mock tumor 612 was referred to as muscular tissue in the software simulation, as it presents relatively high conductivity, typical to tumors. In other words, the mock tumor 612 may be assigned the dielectric properties typical to tumors. For the simulation, the resected vertebrae included cervical vertebrae C5 to thoracic vertebrae T2, the mock tumor 612, and an implant 610. For the simulation, two materials were tested, namely titanium and PEEK. In some embodiments, a “mix material” implant 610 may be simulated. For example, an implant 610 with a non-conductive material “core” coated with a thin layer of conductive material, or the inverse, may be tested in the simulation.

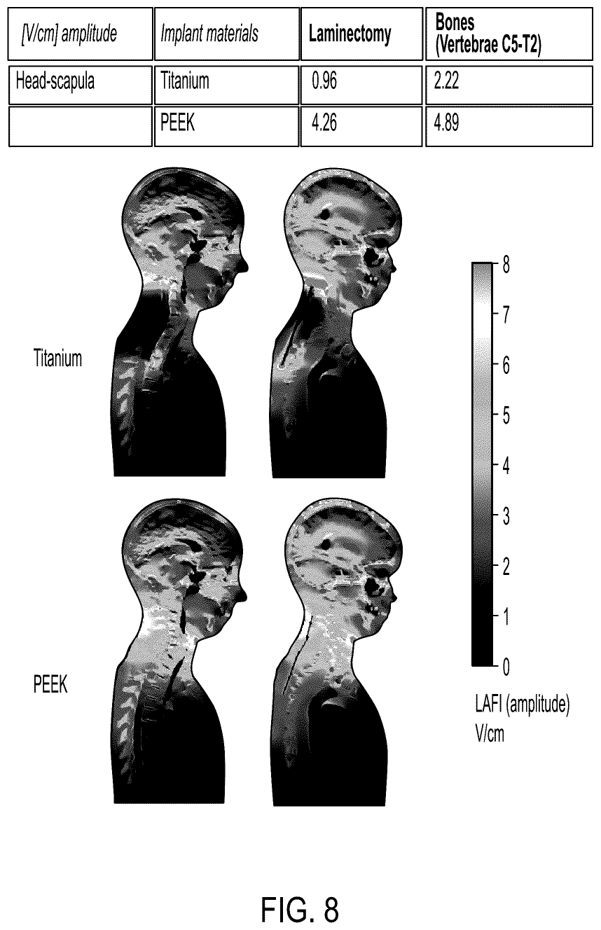

FIG. 8 illustrates the simulation results using the exemplary layout of transducers of FIG. 6. As can be seen, the exemplary layout of transducers provides high dosages of TTFields for both the laminectomy region and a region around vertebrae C5-T2. Further, using PEEK as the material for the implant 610 provides higher dosages of TTFields than using titanium as the material for the implant 610.

### Lower Lumber-Sacral Region-Vertebrae L4-L5

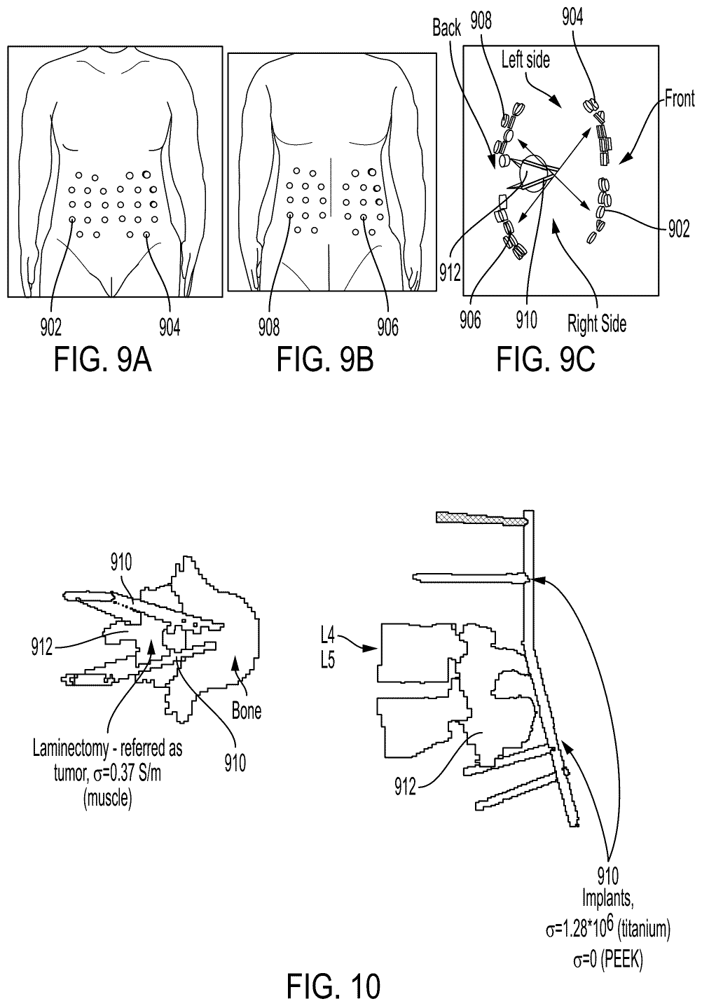

FIG. 9 (which includes FIGS. 9A, 9B, and 9C) illustrates an exemplary layout of transducers to deliver TTFields to a tumor in a lower lumbar region of a subject, in a sacral region of a subject, and/or in a region around vertebrae L4-L5 of a subject.

FIG. 9A illustrates a front view of the subject, FIG. 9B illustrates a back view of the subject, and FIG. 9C illustrates a top view of the four transducers with the subject redacted and an implant 910 and mock tumor 912. The exemplary layout includes four transducers, including: a first transducer 902 located on the right side of the abdomen on the front of the subject; a second transducer 904 located on left side of the abdomen on the front of the subject; a third transducer 906 located on the right side of the lower back of the subject; and a fourth transducer 908 located on left side of the lower back of the subject. When applying TTFields to the subject, AC voltage is alternately applied to a first pair of transducers 902 and 908 and to a second pair of transducers 904 and 906. In the example of FIG. 9, each of the first, second, third, and fourth transducers have 13 electrode elements. In some embodiments, each of the first, second, third, and fourth transducers may have less than or more than 13 electrode elements. In some embodiments, the first, second, third, and fourth transducers may have a different number of electrode elements.

Each of the first, second, third, and fourth transducers may be configured in a variety of shapes and positions to generate an electric field of the desired configuration, direction, and intensity at a target volume to focus treatment. Each of the first, second, third, and fourth transducers may be configured to deliver two perpendicular field directions through a volume of interest. The arrows in FIG. 9C indicate the TTfields generated within the treated tumor for each of the two pairs of transducers.

FIG. 10 illustrates a laminectomy model for use in a computer simulation using the exemplary layout of transducers of FIG. 9. The model appears pixelated due to the discrete computation model used. The electric distribution generated between the transducers may be simulated to determine a desired treatment. For the simulation, the model included a “virtual laminectomy” on the subject's spine. In the resected region, a mock tumor 912 was inserted to mimic typical metastatic growth in the region. The mock tumor 912 was referred to as muscular tissue in the software simulation, as it presents relatively high conductivity, typical to tumors. In other words, the mock tumor 912 may be assigned the dielectric properties typical to tumors. For the simulation, the resected vertebrae included lumbar vertebrae L4 to L5. For the simulation, two materials were tested, namely titanium and PEEK. In some embodiments, a “mix material” implant 910 may be simulated. For example, an implant 910 with a non-conductive material “core” coated with a thin layer of conductive material, or the inverse, may be tested in the simulation.

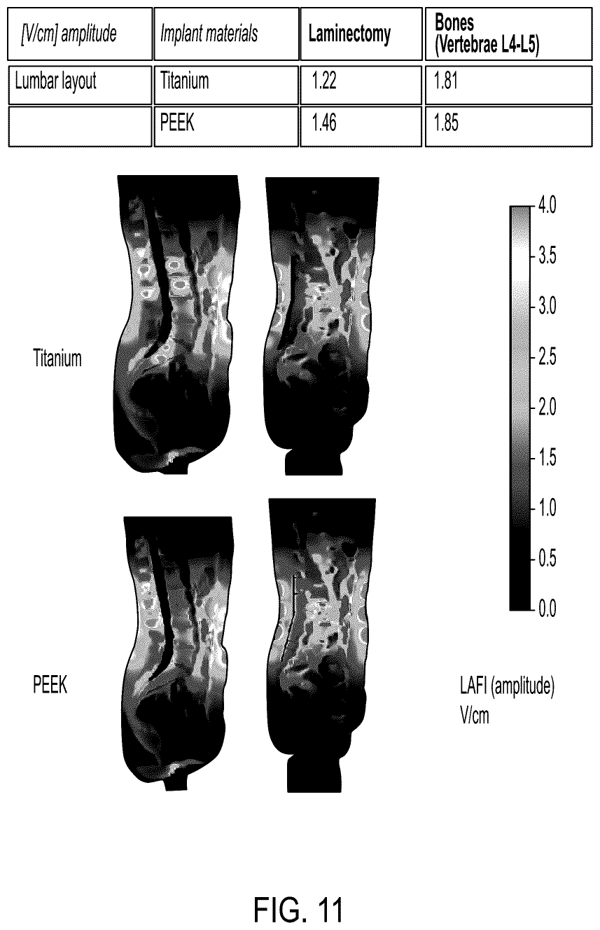

FIG. 11 illustrates the simulation results using the exemplary layout of transducers of FIG. 9. As can be seen, the exemplary layout of transducers provides high dosages of TTFields for both the laminectomy region and a region around vertebrae L4-L5. Further, using PEEK as the material for the implant 910 provides higher dosages of TTFields than using titanium as the material for the implant 910.

In some embodiments, a location of a transducer (for example, one or more of the transducers in FIGS. 1, 4, 6, and 9) on a subject may account for anatomical restrictions. The anatomical restriction may be associated with an anatomical feature of the subject, for example, joints, armpits, nipples, genitals, and the like. The anatomical restriction may be associated with an area of the subject that should be avoided for transducer placement because of discomfort or pain that may be caused to the subject, for example, areas of irritation, wounds, scars, and the like. The anatomical restriction may be determined based on a subject model and/or by observation.

In some embodiments, placement locations for each of the first, second, third, and fourth transducers on a portion of the subject's body may be sought to obtain a desired TTFields therapy. The first, second, third, and fourth transducers may be those discussed herein, for example, with respect to FIGS. 1, 4, 6, and 9. As an example, imaging data, such as MRI imaging data, may be analyzed by a computer processor (such as one or more processors 1502). In the context of the subject's spine, to characterize how electric fields behave and distribute within the human spine, particularly when implants are present, modeling frameworks based on anatomical spine models using Finite Element Method (FEM) simulations may be used. These simulations may yield realistic spine models based on magnetic resonance imaging (MRI) measurements and compartmentalize tissue and implant material types. Each tissue and implant material type may be assigned dielectric properties for relative conductivity and permittivity, and simulations may be run whereby different transducer configurations may be applied to the surface of the model to understand how an externally applied electric field, of preset frequency, will distribute throughout any portion of a subject's body, for example, the spine and/or torso. In doing so, a precise placement of the first, second, third, and fourth transducers on the subject's body may be obtained with respect to the tumor and the spine and/or torso.

### Illustrative Apparatuses

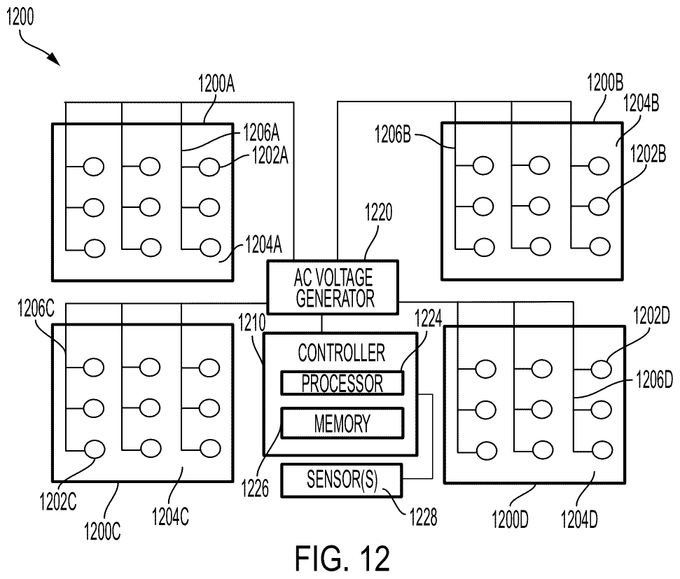

FIG. 12 depicts an example apparatus 1200 to apply alternating electric fields (e.g., TTFields) to a subject according to one or more embodiments discussed herein. The system may be used for treating a target region of a subject's body with an alternating electric field. In an example, the target region may be in the subject's brain, and an alternating electric field may be delivered to the subject's body via two pairs of transducers positioned on a head of the subject's body (such as, for example, in FIG. 14, which has four transducers 1400). In another example, the target region may be in the subject's torso (e.g., in the spine), and an alternating electric field may be delivered to the subject's body via two pairs of transducers positioned on at least one of a thorax, an thoracic-high lumbar, cervical area, sacral region, or one or both thighs of the subject's body. Example locations for placing transducers on a subject are shown in FIGS. 1, 4, 6, and 9. Other transducer placements on the subject's body may be possible.

The example apparatus 1200 depicts an example system having four transducers 1200A-D. Each transducer 1200A-D may include substantially flat electrode elements 1202A-D positioned on a substrate 1204A-D and electrically and physically connected (e.g., through conductive wiring 1206A-D). For each substrate 1204A-D, the respective electrode elements 1202A-D of the substrate may be electrically connected to each other and may be physically connected to their respective substrate 1204A-D. In an example, the electrode elements 1202A-D may be controlled as a collective, such that the electrode elements 1202A-D receive and execute a same instruction signal. In an example, the electrode elements 1202A-D may be individually controlled, such that each electrode element may receive and execute an instruction different from an instruction received and executed by another electrode element.

The substrates 1204A-D may include, for example, cloth, foam, flexible plastic, and/or conductive medical gel. Two transducers (e.g., 1200A and 1200D) may be a first pair of transducers configured to apply an alternating electric field to a target region of the subject's body. The other two transducers (e.g., 1200B and 1200C) may be a second pair of transducers configured to similarly apply an alternating electric field to the target region.

The transducers 1200A-D may be coupled to an alternating current (AC) voltage generator 1220, and the system may further include a controller 1210 communicatively coupled to the AC voltage generator 1220. The controller 1210 may include a computer having one or more processors 1224 and memory 1226 accessible by the one or more processors 1224. The memory 1226 may store instructions that when executed by the one or more processors 1224 control the AC voltage generator 1220 to induce alternating electric fields between pairs of the transducers 1200A-D according to one or more voltage waveforms and/or cause the computer to perform one or more methods disclosed herein. The controller 1210 may monitor operations performed by the AC voltage generator 1220 (e.g., via the processor(s) 1224). One or more sensor(s) 1228 may be coupled to the controller 1210 for providing measurement values or other information to the controller 1210 (e.g., thermistors providing temperature measurements).

In some embodiments, the voltage generation components may supply the transducers 1200A-D with an electrical signal having an alternating current waveform at frequencies in a range from about 50 kHz to about 1 MHz and appropriate to deliver TTFields treatment to the subject's body.

The electrode elements 1202A-D may be capacitively coupled. In one example, the electrode elements 1202A-D are ceramic electrode elements coupled to each other via conductive wiring 1206A-D. When viewed in a direction perpendicular to its face, the ceramic electrode elements may be circular shaped or non-circular shaped. In other embodiments, the array of electrode elements are not capacitively coupled, and there is no dielectric material (such as ceramic, or high dielectric polymer layer) associated with the electrode elements.

The structure of the transducers 1200A-D may take many forms. The transducers may be affixed to the subject's body or attached to or incorporated in clothing covering the subject's body. The transducer may include suitable materials for attaching the transducer to the subject's body. For example, the suitable materials may include cloth, foam, flexible plastic, and/or a conductive medical gel. The transducer may be conductive or non-conductive.

The transducer may include any desired number of electrode elements (e.g., one or more electrode elements). For example, the transducer may include one, two, three, four, five, six, seven, eight, nine, ten, or more electrode elements (e.g., thirteen electrode elements or twenty electrode elements). Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein.

In some embodiments, an AC signal may be capacitively coupled into the subject's body. In some embodiments, at least one electrode element of the first, the second, the third, or the fourth transducer may include at least one ceramic disk that is adapted to generate an alternating electric field. In some embodiments, at least one electrode element of the first, the second, the third, or the fourth transducer includes a polymer film that is adapted to generate an alternating electric field.

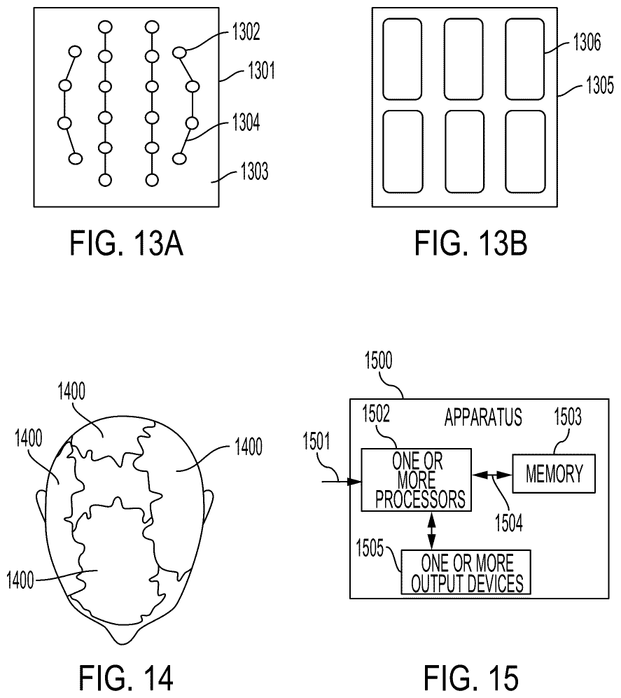

FIG. 13A depicts a schematic view of an exemplary design of a transducer for applying alternating electric fields. The transducer 1301 includes twenty electrode elements 1302, which are positioned on the substrate 1303, and the electrode elements 1302 are electrically and physically connected to one another through a conductive wiring 1304. In some embodiments, the electrode elements 1302 may include a ceramic disk.

FIG. 13B depicts a schematic view of an exemplary design of a transducer for applying alternating electric fields. The transducer 1305 may include one or more substantially flat electrode elements 1306. In some embodiments, the electrode elements 1306 are non-ceramic dielectric materials positioned over a plurality of flat conductors. Examples of non-ceramic dielectric materials positioned over flat conductors may include polymer films disposed over pads on a printed circuit board or over substantially planar pieces of metal. In an embodiment, such polymer films have a high dielectric constant, such as, for example, a dielectric constant greater than 10. In some embodiments, the electrode elements 1306 may have various shapes. For example, the electrode elements may be triangular, rectangular, circular, oval, ovaloid, ovoid, or elliptical in shape or substantially triangular, substantially rectangular, substantially circular, substantially oval, substantially ovaloid, substantially ovoid, or substantially elliptical in shape. In some embodiments, each of electrode elements 1306 may have a same shape, similar shapes, and/or different shapes.

FIG. 15 depicts an example computer apparatus for use with the embodiments herein. As an example, obtaining a three-dimensional model of a subject and/or simulating such a three-dimensional model to determine placement locations for transducers on a subject may be performed by a computer, such as computer apparatus 1500. As an example, the apparatus 1500 may be a computer to implement certain inventive techniques disclosed herein. As an example, the apparatus 1500 may be a controller apparatus to apply the alternating electric fields (e.g., TTFields) with modulated electric fields for the embodiments herein. The controller apparatus 1500 may be used as the controller 1210 of FIG. 12. As an example, the apparatus 1500 may be used to perform simulations of delivering alternating electric fields (e.g., TTFields) to a subject based on a model of electrical conductivity in a portion of the subject and a number of possible layouts of transducers for the subject and then to select one or more recommended layouts of transducers to use for the subject based on the simulation results. The apparatus 1500 may include one or more processors 1502, memory 1503, one or more input devices, and one or more output devices 1505.

In one example, based on input 1501, the one or more processors generate control signals to control the voltage generator to implement an embodiment of the present disclosure. In one example, the input 1501 may be user input. In another example, the input 1501 may be from another computer in communication with the controller apparatus 1500. The input 1501 may be received in conjunction with one or more input devices (not shown) of the apparatus 1500.

The memory 1503 may be accessible by the one or more processors 1502 (e.g., via a link 1504) so that the one or more processors 1502 may read information from and write information to the memory 1503. The memory 1503 may store instructions that when executed by the one or more processors 1502 implement one or more embodiments of the present disclosure. The memory 1503 may be a non-transitory computer readable medium (or a non-transitory processor readable medium) containing a set of instructions thereon for selecting at least one transducer layout for delivering tumor treating fields to a subject, wherein when executed by a processor (such as one or more processors 1502), the instructions cause the processor to perform one or more methods discussed herein.

The one or more output devices 1505 may provide the status of the operation of the invention, such as transducer selection, voltages being generated, and other operational information. The output device(s) 1505 may provide visualization data according to certain embodiments described herein.

The apparatus 1500 may be an apparatus for selecting at least one transducer layout for delivering tumor treating fields to a subject, the apparatus including: one or more processors (such as one or more processors 1502); and memory (such as memory 1503) accessible by the one or more processors, the memory storing instructions that when executed by the one or more processors, cause the apparatus to perform one or more methods described herein.

### Illustrative Methods

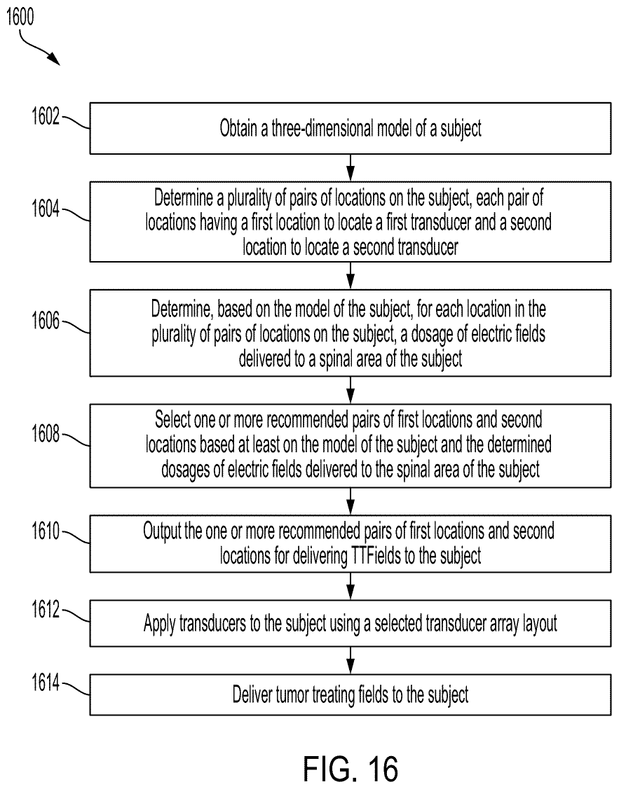

FIG. 16 depicts an example computer-implemented method 1600 for selecting at least one transducer array layout for delivering TTFields to the subject according to one or more embodiments described herein. The method 1600 may be implemented by a computer, the computer including one or more processors and memory accessible by the one or more processors, the memory storing instructions that when executed by the one or more processors cause the computer to perform the steps of the method 1600. The method 1600 may be implemented by a computer, such as apparatus 1500. In some embodiments, steps 1602 to 1610 and step 1614 may be performed by a computer. In some embodiments, steps 1602 to 1610 may be performed by a first computer, and step 1614 may be performed by a second computer. Modifications, additions, or omissions may be made to method 6100.

The method 1600 includes, at step 1602, obtaining a three-dimensional (3D) model of the subject. In some embodiments, the 3D model may be a model of electrical conductivity in a portion of the subject. The 3D model includes voxels. Each voxel of the model may be assigned a type of tissue (e.g., bone, organs, fluid, skin, or tumor) and/or an electrical conductivity associated with the type of tissue. In one example, the model of the subject may represent a head of the subject. In another example, the model of the subject may represent a torso of the subject. Other body parts of the subject may be represented in the model of the subject in other embodiments.

In some embodiments, the model of the subject may be of a portion of the subject, such as, for example, the spine and/or torso of the subject. The model of the subject may include an implant comprised of a non-conductive material. The model of the subject may include an implant comprised of a non-conductive material in a spinal area of the subject. As an example, the non-conductive material of the implant is comprised of polyether ether ketone (PEEK). The model of the subject may include an implant comprised of a non-conductive material and a conductive material. The model of the subject may include an implant in the spinal area of the subject, where the implant is comprised of a non-conductive material and a conductive material. As an example, the non-conductive material of the implant is comprised of polyether ether ketone (PEEK) and the conductive material of the implant is comprised of titanium.

In some embodiments, the model may be obtained using image data, for example, via a computer identifying the different types of tissue from the image data. In some embodiments, data within the medical images can be correlated to tissue type (and eventually conductivity) using segmentation, voxel intensity, relative location within the body, and/or the like including combinations and/or multiples thereof. The image data may include one or more medical images of a portion of the subject's body (e.g., X-ray images, magnetic resonance imaging (MRI), computerized tomography (CT) images, ultrasound images, or any image providing an internal view of the subject's body). Each medical image may include an outer shape of a portion of the subject and a region corresponding to a region of interest (e.g., a tumor) within the subject. The three-dimensional model may be obtained, for example, from computer memory locally or over a network.

At step 1604, the method 1600 may include determining a plurality of pairs of locations on the subject, each pair of locations having a first location to locate a first transducer and a second location to locate a second transducer. A plurality of transducer layouts for delivering TTFields to the subject may be determined, and each transducer layout may have an associated location on the subject. In an example, the transducer layouts in the plurality of transducer layouts may differ by at least one of location on the subject, size of the transducer, shape of the transducer, number of electrodes of the transducer, size of electrodes of the transducer, or shape of electrodes of the transducer. In an example, step 1604 includes determining pairs of transducer layouts. In an example, step 1604 includes determining two pairs of transducer layouts. In some embodiments, the plurality of pairs of locations on the subject determined in step 1604 may be possible locations for placing transducers on the subject to administer TTFields, and method 1600 may determine recommended pairs of locations from these possible pairs of locations and output one or more recommended pairs of locations in step 1610. In some embodiments, the transducer layouts may be one or more of those described herein with reference to FIGS. 1, 4, 6, and 9.

At step 1606, the method 1600 may include determining, by the one or more processors and based on the 3D model, for each location in the plurality of pairs of locations on the subject, a dosage of electric fields delivered to a spinal area of the subject. Tumor treating fields dosages may be calculated for the plurality of transducer layouts. Step 1606 may include calculating a TTFields dosage for each determined transducer layout. In an example, the TTFields dosage may be based on a local average field intensity and/or a local power density within a target range.

As an example, calculating the dosage of TTFields treatment is further described in more detail in U.S. Patent Application Publication No. 2020/0023179, entitled “USING POWER LOSS DENSITY AND RELATED MEASURES TO QUANTIFY THE DOSE OF TUMOR TREATING FIELDS (TTFIELDS)” and U.S. Patent Publication No. 2021/0196943, entitled “METHODS, SYSTEMS, AND APPARATUSES FOR FAST APPROXIMATION OF ELECTRIC FIELD DISTRIBUTION,” the entire contents of both of which are incorporated by reference herein. It should be appreciated that these calculations in step 1606 involve solving complex algorithms using large data sets associated with the subject and, as such, require the use of a computer apparatus, as the human mind is not capable of performing the required calculations.

At step 1608, the method 1600 may include selecting one or more recommended pairs of first locations and second locations based at least on the model of electrical conductivity and the determined dosages of electric fields delivered to the spinal area of the subject. At least one transducer layout may be selected for delivering TTFields to the subject, wherein the at least one transducer layout may be selected from the plurality of transducer layouts determined at step 1604.

At step 1610, the method 100 may include outputting the one or more recommended pairs of first locations and second locations for delivering TTFields to the subject. The recommended pairs of first locations and second locations may include associated transducers, which may or may not be the same size, be the same shape, have the same number of electrode elements, have the same size of electrode elements, and/or have the same shape of electrode elements. The selected at least one transducer layout may be output. The transducer location(s) are locations on the subject where the transducers are to be placed for applying TTFields to the subject. In some embodiments, using the recommended pairs of locations, a physician may select certain pairs of locations and associated transducers that achieve a minimum dosage for treating the tumor of the subject.

At step 1612, the method 1600 may include applying transducers to the subject using a selected transducer layout. The selected transducer layout may be one of the recommended transducer layouts from step 1610. More details of step 1612 are described herein with reference to FIGS. 1, 4, 6, 9, and 12.

At step 1614, the method 1600 may include delivering the TTFields to the subject based on the selected transducer layout. More details of step 1614 are described herein with reference to FIG. 12.

### ILLUSTRATIVE EMBODIMENTS

The invention includes other illustrative embodiments (“Embodiments”) as follows

Embodiment 1. A method of applying tumor treating fields to a subject, the method comprising: locating a plurality of transducers on the subject, the plurality of transducers being located to focus electric fields around a spinal area of the subject; and alternately inducing a first electric field for a first set of the transducers and a second electric field for a second set of the transducers.

Embodiment 2. The method of Embodiment 1, wherein the plurality of transducers are located to focus electric fields on at least one of a thoracic-high lumbar region of the subject or a region around vertebrae T2-LA of the subject.

Embodiment 3. The method of Embodiment 1, wherein the plurality of transducers are located to focus electric fields on at least one of a cervical region of the subject, a cervical-high thoracic region of the subject, or a region around vertebrae C5-T2 of the subject.

Embodiment 4. The method of Embodiment 1, wherein the plurality of transducers are located to focus electric fields on at least one of a lower lumbar region of the subject, a sacral region of the subject, or a region around vertebrae L4-L5 of the subject.

Embodiment 5. The method of Embodiment 1, wherein the subject has a non-conductive implant in the spinal area where the electric fields are focused by the plurality of transducers.

Embodiment 6. The method of Embodiment 1, wherein the non-conductive implant is comprised of polyether ether ketone (PEEK).

Embodiment 7. The method of Embodiment 1, wherein the non-conductive implant does not include titanium.

Embodiment 8. The method of Embodiment 1, wherein the plurality of transducers include at least two pairs of transducers.

Embodiment 9. A system to apply tumor treating fields to a subject, the system comprising: a first transducer adapted to be located at a first location of the subject's body; a second transducer adapted to be located at a second location of the subject's body; a third transducer adapted to be located at a third location of the subject's body; a fourth transducer adapted to be located at a fourth location of the subject's body; an alternating current (AC) voltage generator adapted to be coupled to the first, second, third, and fourth transducers and capable of providing first, second, third, and fourth voltages respectively to the first, second, third, and fourth transducers; and a controller coupled to the AC voltage generator, the controller comprising one or more processors and memory accessible by the one or more processors, the memory storing instructions that when executed by the one or more processors, cause the controller to: instruct the AC voltage generator to generate the first, second, third, and fourth voltages to alternately induce a first electric field in the subject between the first and second transducers and a second electric field in the subject between the third and fourth transducers, wherein the alternating first and second electric fields are focused on a spinal area of the subject.

Embodiment 10. The system of Embodiment 9, wherein the first transducer, second transducer, third transducer, and fourth transducer are located to focus the alternating first and second electric fields on at least one of a thoracic-high lumbar region of the subject or a region around vertebrae T2-LA of the subject.

Embodiment 11. The system of Embodiment 9, wherein the first transducer, second transducer, third transducer, and fourth transducer are located to focus the alternating first and second electric fields on at least one of a cervical region of the subject, a cervical-high thoracic region of the subject, or a region around vertebrae C5-T2 of the subject.

Embodiment 12. The system of Embodiment 9, wherein the first transducer, second transducer, third transducer, and fourth transducer are located to focus the alternating first and second electric fields on at least one of a lower lumbar region of the subject, a sacral region of the subject, or a region around vertebrae L4-L5 of the subject.

Embodiment 13. The system of Embodiment 9, wherein the subject has a non-conductive implant in the spinal area where the alternating first and second electric fields are focused.

Embodiment 14. The system of Embodiment 13, wherein the non-conductive implant is comprised of polyether ether ketone (PEEK).

Embodiment 14A. The system of Embodiment 9, wherein the AC voltage generator supplies an electrical signal having an alternating current waveform at frequencies ranging from at least 50 kHz to at most 1 MHz.

Embodiment 15. A computer-implemented method to determine placement of transducers on a subject for inducing tumor treating fields, the computer comprising one or more processors and memory accessible by the one or more processors, the memory storing instructions that when executed by the one or more processors cause the computer to perform the method, the method comprising: obtaining a model of electrical conductivity in a portion of the subject, wherein the portion of the subject includes an implant comprised of a non-conductive material; selecting a plurality of pairs of locations on the subject, each pair of locations having a first location to locate a first transducer and a second location to locate a second transducer; determining, by the one or more processors and based on the model of electrical conductivity, for each location in the plurality of pairs of locations on the subject, a dosage of electric fields delivered to a spinal area of the subject; selecting one or more recommended pairs of first locations and second locations based at least on the model of electrical conductivity and the determined dosages of electric fields delivered to the spinal area of the subject; and outputting the one or more recommended pairs of first locations and second locations.

Embodiment 16. The computer-implemented method of Embodiment 15, wherein the non-conductive material of the implant is comprised of polyether ether ketone (PEEK).

Embodiment 17. The computer-implemented method of Embodiment 15, wherein the implant is located in a spinal area of the subject.

Embodiment 18. The computer-implemented method of Embodiment 15, wherein the one or more recommended pairs of first locations and second locations are located to focus tumor treating fields on at least one of a thoracic-high lumbar region of the subject or a region around vertebrae T2-L4 of the subject.

Embodiment 19. The computer-implemented method of Embodiment 15, wherein the one or more recommended pairs of first locations and second locations are located to focus electric fields on at least one of a cervical region of the subject, a cervical-high thoracic region of the subject, or a region around vertebrae C5-T2 of the subject.

Embodiment 20. The computer-implemented method of Embodiment 15, wherein the one or more recommended pairs of first locations and second locations are located to focus electric fields on at least one of a lower lumbar region of the subject, a sacral region of the subject, or a region around vertebrae L4-L5 of the subject.

Optionally, for each embodiment described herein, the voltage generation components supply the transducers with an electrical signal having an alternating current waveform at frequencies in a range from about 50 kHz to about 1 MHz and appropriate to deliver TTFields treatment to the subject's body.

Embodiments illustrated under any heading or in any portion of the disclosure may be combined with embodiments illustrated under the same or any other heading or other portion of the disclosure unless otherwise indicated herein or otherwise clearly contradicted by context.

For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A method of applying tumor treating fields to a subject, the method comprising:
locating a plurality of transducers on the subject, the plurality of transducers being located to focus electric fields around a spinal area of the subject; and
alternately inducing a first electric field for a first set of the transducers and a second electric field for a second set of the transducers.

2. The method of claim 1, wherein the plurality of transducers are located to focus electric fields on at least one of a thoracic-high lumbar region of the subject or a region around vertebrae T2-L4 of the subject.

3. The method of claim 1, wherein the plurality of transducers are located to focus electric fields on at least one of a cervical region of the subject, a cervical-high thoracic region of the subject, or a region around vertebrae C5-T2 of the subject.

4. The method of claim 1, wherein the plurality of transducers are located to focus electric fields on at least one of a lower lumbar region of the subject, a sacral region of the subject, or a region around vertebrae L4-L5 of the subject.

5. The method of claim 1, wherein the subject has a non-conductive implant in the spinal area where the electric fields are focused by the plurality of transducers.

6. The method of claim 1, wherein the non-conductive implant is comprised of polyether ether ketone (PEEK).

7. The method of claim 1, wherein the non-conductive implant does not include titanium.

8. The method of claim 1, wherein the plurality of transducers include at least two pairs of transducers.

9. A system to apply tumor treating fields to a subject, the system comprising:
a first transducer adapted to be located at a first location of the subject's body;
a second transducer adapted to be located at a second location of the subject's body;
a third transducer adapted to be located at a third location of the subject's body;
a fourth transducer adapted to be located at a fourth location of the subject's body;
an alternating current (AC) voltage generator adapted to be coupled to the first, second, third, and fourth transducers and capable of providing first, second, third, and fourth voltages respectively to the first, second, third, and fourth transducers; and
a controller coupled to the AC voltage generator, the controller comprising one or more processors and memory accessible by the one or more processors, the memory storing instructions that when executed by the one or more processors, cause the controller to:
instruct the AC voltage generator to generate the first, second, third, and fourth voltages to alternately induce a first electric field in the subject between the first and second transducers and a second electric field in the subject between the third and fourth transducers, wherein the alternating first and second electric fields are focused on a spinal area of the subject.

10. The system of claim 9, wherein the first transducer, second transducer, third transducer, and fourth transducer are located to focus the alternating first and second electric fields on at least one of a thoracic-high lumbar region of the subject or a region around vertebrae T2-L4 of the subject.

11. The system of claim 9, wherein the first transducer, second transducer, third transducer, and fourth transducer are located to focus the alternating first and second electric fields on at least one of a cervical region of the subject, a cervical-high thoracic region of the subject, or a region around vertebrae C5-T2 of the subject.

12. The system of claim 9, wherein the first transducer, second transducer, third transducer, and fourth transducer are located to focus the alternating first and second electric fields on at least one of a lower lumbar region of the subject, a sacral region of the subject, or a region around vertebrae L4-L5 of the subject.

13. The system of claim 9, wherein the subject has a non-conductive implant in the spinal area where the alternating first and second electric fields are focused.

14. The system of claim 13, wherein the non-conductive implant is comprised of polyether ether ketone (PEEK).

15. A computer-implemented method to determine placement of transducers on a subject for inducing tumor treating fields, the computer comprising one or more processors and memory accessible by the one or more processors, the memory storing instructions that when executed by the one or more processors cause the computer to perform the method, the method comprising:
obtaining a model of electrical conductivity in a portion of the subject, wherein the portion of the subject includes an implant comprised of a non-conductive material;
selecting a plurality of pairs of locations on the subject, each pair of locations having a first location to locate a first transducer and a second location to locate a second transducer;
determining, by the one or more processors and based on the model of electrical conductivity, for each location in the plurality of pairs of locations on the subject, a dosage of electric fields delivered to a spinal area of the subject;
selecting one or more recommended pairs of first locations and second locations based at least on the model of electrical conductivity and the determined dosages of electric fields delivered to the spinal area of the subject; and
outputting the one or more recommended pairs of first locations and second locations.

16. The computer-implemented method of claim 15, wherein the non-conductive material of the implant is comprised of polyether ether ketone (PEEK).

17. The computer-implemented method of claim 15, wherein the implant is located in a spinal area of the subject.

18. The computer-implemented method of claim 15, wherein the one or more recommended pairs of first locations and second locations are located to focus tumor treating fields on at least one of a thoracic-high lumbar region of the subject or a region around vertebrae T2-L4 of the subject.

19. The computer-implemented method of claim 15, wherein the one or more recommended pairs of first locations and second locations are located to focus electric fields on at least one of a cervical region of the subject, a cervical-high thoracic region of the subject, or a region around vertebrae C5-T2 of the subject.

20. The computer-implemented method of claim 15, wherein the one or more recommended pairs of first locations and second locations are located to focus electric fields on at least one of a lower lumbar region of the subject, a sacral region of the subject, or a region around vertebrae L4-L5 of the subject.
